(19) United States
(12) Patent Application Publication
(10) Pub. No.: US 2011/0237842 A1
(43) Pub. Date: Sep. 29, 2011

(54) UV REACTOR FOR CHEMICAL REACTIONS AND USE THEREOF

(75) Inventors: Ernst Martin Billing, Bielefeld (DE); Friedhelm Krüger, Lemgo (DE)

(73) Assignee: ITT MANUFACTURING ENTERPRISES, INC., WILMINGTON, DE (US)

(21) Appl. No.: 13/124,212

(22) PCT Filed: Jun. 2, 2009
(86) PCT No.: PCT/EP09/03914

§ 371 Date: Jun. 14, 2011

(30) Foreign Application Priority Data
Oct. 17, 2008 (DE) 10 2008 051 798.4

(51) Int. Cl. C07C 17/00; B01J 19/12; B01J 19/08

(52) U.S. Cl. 570/101; 422/186.3; 422/186.04

(57) ABSTRACT

An ultraviolet (UV) reactor for carrying out chemical reactions in a pumpable medium by means of UV. The pumpable medium may also be, where appropriate, a multi-phase medium. The UV reactor has a reactor chamber through which the medium can flow In a direction of flow from an inlet to an outlet. The reactor chamber is penetrated by a number of UV transparent jacket tubes, which are arranged one behind the other in the direction of flow. UV emitters are arranged within the jacket tubes for emitting UV radiation into the reactor chamber. The jacket tubes are arranged one behind the other and are interlocked against one another at an angle αin the circumferential direction of the reactor chamber.

### CROSS REFERENCE TO RELATED APPLICATIONS

This application is the U.S. National Phase Application of PCT International Application No. PCT/EP2009/003914, filed Jun. 2, 2009, which claims priority to German Patent Application No. 10 2008 051 798.4, filed Oct. 17, 2008, the contents of such applications being incorporated by reference herein.

### FIELD OF THE INVENTION

The present invention relates to a Ultraviolet (UV) reactor for carrying out chemical reactions.

### BACKGROUND OF THE INVENTION

It is known to add an oxidising means, such as ozone or H₂O₂, in chemical reactions, if oxidation is intended. With substances which are difficult to oxidise, it is further known to additionally beam UV radiation into the reaction chamber in order to create radicals. In this way, for example, halogenated hydrocarbons and residues of pharmaceutical substances can be oxidised and hence rendered harmless.

With the known devices, a number of UV emitters radiate into the liquid or gaseous medium. The emitters are arranged parallel or transverse to the direction of flow of the medium for this purpose. They can be arranged inside a reaction chamber, but with UV transparent reaction chambers they can also be arranged outside the medium.

The effectiveness of the device depends on how well the oxidation means and the medium to be treated are intermixed and how homogenously the irradiation into the medium takes place. The concentration of the oxidation means should, as far as possible, be constant over the entire medium volume to be treated and also each partial volume of the medium should receive the same UV dose. The less these requirements are fulfilled, the more oxidation means and UV radiation have to be supplied in excess.

### SUMMARY OF THE INVENTION

Therefore, it is an object of the present invention to create a device for carrying out chemical reactions under oxidising conditions, which has the best possible effectiveness.

Due to the fact that UV emitters, arranged one behind the other in the direction of flow, are staggered against one another at an angle with respect to the radial direction, the probability drops that partial volumes of the medium to be treated pass through the device on a flow path which does not have sufficient UV intensity and as a result no chemical reactions are induced there. In particular, multi-phase, pumpable media can also thereby be effectively treated.

A good effect is obtained if the angle α is 15° to 45°, preferably 30° . The angle α, according to the embodiment, can, for example, be chosen as a function of the diameter of the reactor.

Emitters with a greater discharge length can be used if the jacket tubes are inclined at an angle β of 30° to 70° with respect to the radial direction of the reactor chamber.

All possible flow paths can be extensively irradiated if at least two groups of jacket tubes are provided, each of which one jacket tube is arranged next to a jacket tube of the other group with respect to the centre axis of the reactor chamber, and wherein the groups in each case form a separate helical row. Three or more emitters can also be arranged next to one another in a radial plane for a particularly high flow rate and/or media with particularly low UV transmission. The areas close to the wall of the reactor chamber are also in the process reached if the jacket tubes are arranged at a distance from the centre axis.

The outcome will be particularly good if the groups are at different distances from the centre axis, that is to say, a first group is a long distance away and a second group is a short distance away. In addition, the first group can be aligned at a large angle β of 50° to 70° and the second group can be aligned at a smaller angle β of 30° to 49° to the radial direction, so that both groups can be equipped with the same emitters.

Preferably, the larger distance can be more than 60% of the radius of the pump pipe and the smaller distance can be less than 40% of the radius of the reactor chamber. In particular, the one distance can be 75% of the radius of the reactor chamber and the second distance can be 20% of the radius of the reactor chamber. The formation of flow paths with unwanted high flow speed or low intensity can be prevented if the axial distance within a group is also varied, for example by the first group on average having a distance of 60% of the radius, which, however, varies by +/− 10%, while the second group on average has a distance of 20% of the radius, which, likewise, varies by +/− 10% of the radius.

A particularly favourable relation between the number of emitters used and the achieved effect results if each of the groups of jacket tubes comprises in total 12 jacket tubes.

Finally, it is advantageous to use a device according to aspects of the invention for handling inert hydrocarbons, such as halogenated hydrocarbons.

### BRIEF DESCRIPTION OF THE DRAWINGS

The invention is best understood from the following detailed description when read in connection with the accompanying drawings. Included in the drawings is the following figures:

FIG. 1: shows a reactor having parallel arranged emitters according to the prior art;

FIG. 2: shows a reactor according to aspects of the invention in a schematic, perspective illustration;

FIG. 3: shows another reactor in a front view in the direction of flow;

FIG. 4: shows the reactor from FIG. 3 in a longitudinal section;

FIG. 5: shows a reactor having helically arranged UV emitters and a continuous diameter change in the inlet and outlet areas in a cross-section from the side;

FIG. 6: shows a reactor similar to FIG. 5 having a guide vane arrangement in the inlet area;

FIG. 7: shows a reactor having a discontinuous cross-section change in the inlet area; and

FIG. 8: shows a reactor having a built-in device for homogenising the flow.

### DETAILED DESCRIPTION OF THE PREFERRED EMBODIMENTS

In order to bring about chemical reactions by formation of radicals in a flowing medium, for example water, a minimum UV dose needs to be supplied to the medium. Therefore, to get good productivity, the aim is to achieve a high UV intensity at the site of the irradiation, i.e. in the reactor chamber. This intensity is produced by a number of high-performance UV emitters. The emitters themselves are arranged in jacket tubes. These jacket tubes are made of quartz and penetrate the reactor chamber in such a way that they are inserted into the wall in a sealing manner. The emitters are then in turn inserted into the jacket tubes, so that they do not come into contact with the medium, but can emit their radiation output to the medium through the jacket tube.

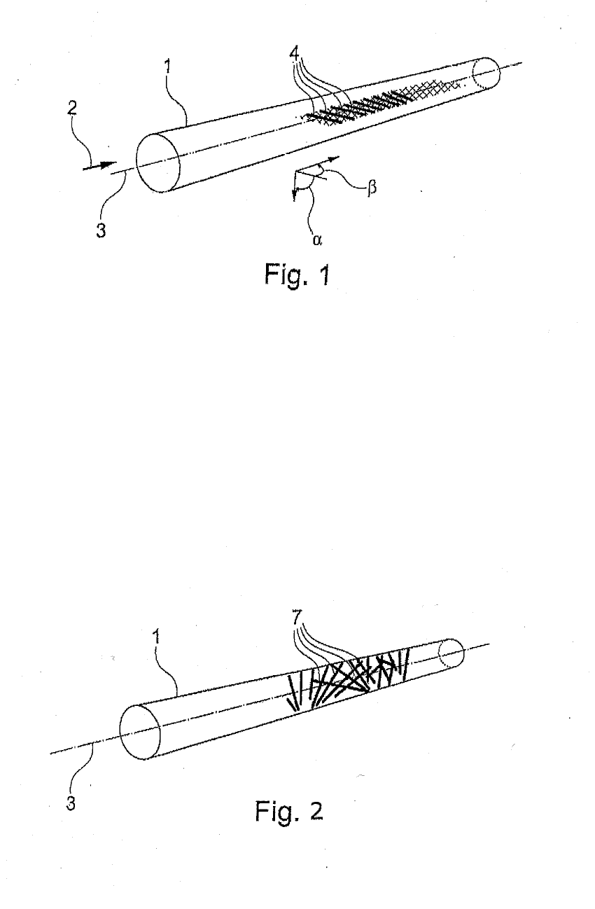

Firstly, the prior art will be explained with the aid of FIG. 1. FIG. 1 shows a tube designed as a reactor chamber having an essentially circular cross-section. The direction of flow runs in the longitudinal direction of the reactor chamber 1, which is indicated by flow arrow 2. A symmetry axis 3 denotes the centre axis of the reactor chamber 1 and represents the rotational symmetry of the arrangement. It is appropriate to define two angles, that is to say, firstly an angle α, which is measured from a horizontally aligned radius in the circumferential direction and in the clockwise direction, and secondly an angle β, which is measured from a radius in the direction of the symmetry axis 3.

A number of UV emitters are attached inside the reactor chamber 1, which are aligned transverse to the direction of flow 2. They are illustrated horizontally in FIG. 1 and so lie in a plane with respect to the centre axis 3. The emitters 4 are arranged in the area of the greatest diameter of the reactor chamber 1. In terms of the angle definition explained above, the angle α is 0° and the angle β is likewise 0°. The individual emitters 4 lie exactly transverse to the centre axis 3 which runs through them.

In the embodiment according to FIG. 1, in practice what happens is that flow paths are formed above and below the emitters 4, in which the UV dose is relatively low, so that an effective reaction can only be achieved with a very high output from the emitters 4. Here, the present invention is now applied, in which an emitter arrangement is chosen which allows practically every possible flow path in its course through the reactor chamber to encounter a UV emitter at least once and therefore, in addition to uniform irradiation, also promotes intermixing of the flowing medium.

An exemplary embodiment of this invention is firstly shown in an illustration in FIG. 2 which corresponds to FIG. 1. FIG. 2 shows the reactor chamber 1 having a number of emitters 7 which in each case are offset by an angle α in relation to one another. The angle α in this illustration is about 30°. In this exemplary embodiment, the distance d for in each case two emitters arranged next to one another is the same.

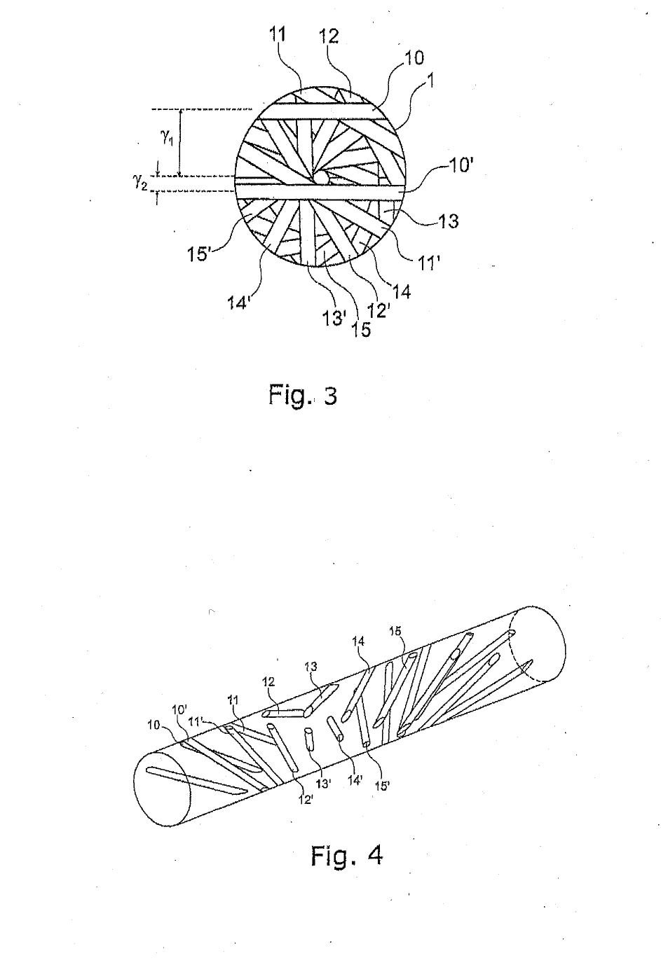

FIG. 3 shows another exemplary embodiment, this time in front view in the direction of the centre axis 3 of the reactor chamber 1. The illustration shows a plurality of jacket tubes which are numbered consecutively from front to back in the direction of flow. Two jacket tubes 10 and 10′ lie in the first plane, the second plane behind it is formed by two jacket tubes 11 and 11′, the third plane by the jacket tubes 12 and 12′, and so on. The term “plane” in this connection is not to be understood strictly as a radial plane, but as the area in which two emitters lie next to one another with respect to the direction of flow of the pumped medium.

It can be identified that the jacket tubes 10, 11, 12, 13, etc. are at a distance r1 from the centre axis 3, which is approximately 75% of the radius of the reactor chamber 1. The distance of the jacket tubes 10′, 11′, 12′, 13′, etc. from the centre axis 3 of the reactor chamber 1 is approximately 18% of the radius of the reactor chamber 1.

While, in the exemplary embodiment according to FIG. 2, the distance in each case between two emitters lying radially next to one another on the centre axis 3 is the same, an exemplary embodiment is shown in FIG. 3 in which the distance between the two emitters lying next to one another is different. This exemplary embodiment is currently preferred.

Considered in the direction of flow of the medium to be irradiated, the arrangement according to FIG. 3 produces a kind of double helix or super helix.

In the exemplary embodiment according to FIG. 3, the chord length of the jacket tubes 10, 11, 12, 13, etc. which is available is shorter than that of the jacket tubes 10′, 11′, 12′, 13′, etc. This is compensated by different angles β with respect to the longitudinal axis 3 of the reactor jacket tube 1, as can be seen from FIG. 4.

FIG. 4 shows, in a schematic illustration, a perspective illustration of the reactor chamber 1 having jacket tubes 11 to 15 and 11′ to 15′ arranged inside it in the configuration corresponding to FIG. 3. The angle β of the jacket tubes 10, 11, 12, 13, etc. is 60° and that of the jacket tubes 10′, 11′, 12′, 13′, etc. lying closer to the axis 3 is 40°. The length of the jacket tubes which is available for emitting UV radiation into the medium is thereby in each case approximately equal.

Here, it is only schematically illustrated that the jacket tubes of the emitters penetrate the wall of the reactor chamber 1 and hence are accessible from the outside. The UV emitters themselves are then inserted into these jacket tubes, so that they can emit their radiation output to the flowing medium inside the reactor chamber 1.

The jacket tubes can also be designed in such a way that they only penetrate the wall of the reactor chamber at one end. This end then holds the mechanical connection and the sealing with the reactor chamber, as well as the electrical and mechanical connections of the emitter. The other end projects freely into the reactor chamber like a finger.

To compare the degrees of effectiveness of the various emitter arrangements in the reactor chamber, calculations were carried out using the Computational Fluid Dynamics (CFD) method. The calculations show a superior UV irradiation of the medium applying the exemplary embodiment according to FIGS. 3 and 4, in which the emitters are arranged in two helically twisted rows, wherein the two rows are at a different distance r₁ and r₂ from the centre axis of the reactor chamber 1, emitters, arranged one behind the other in each case, have an angle α of 30° in relation to one another and the emitter row arranged closer to the centre axis is inclined at an angle β=40° against the radial direction, while the emitter row further away from the centre axis is inclined at an angle β=60° against the radial direction.

While in the above description the design was outlined based on a straight, cylindrical tube for the reactor chamber 1, the reactor chamber can also be twisted, angled or provided with another cross-section. The arrangement of the emitters in the reactor chamber must then be adapted accordingly.

Instead of the described uniformly coiled exemplary embodiment with parallel emitter pairs, the emitters can also be aligned differently, e.g. the emitter pairs can also be offset in relation to one another in the direction of flow, the emitter pairs can have a non-parallel relationship in one plane in the direction of flow and these same emitter pairs can have different angles β.

However, what is similarly vital, in common with uniform and effective irradiation, is that the medium is uniformly intermixed with possibly added oxidation means and other reagents.

To that end, fluidic arrangements at the inlet and/or at the outlet to the reactor chamber are advantageous. Such exemplary embodiments are outlined in the following FIGS. 5 to 8.

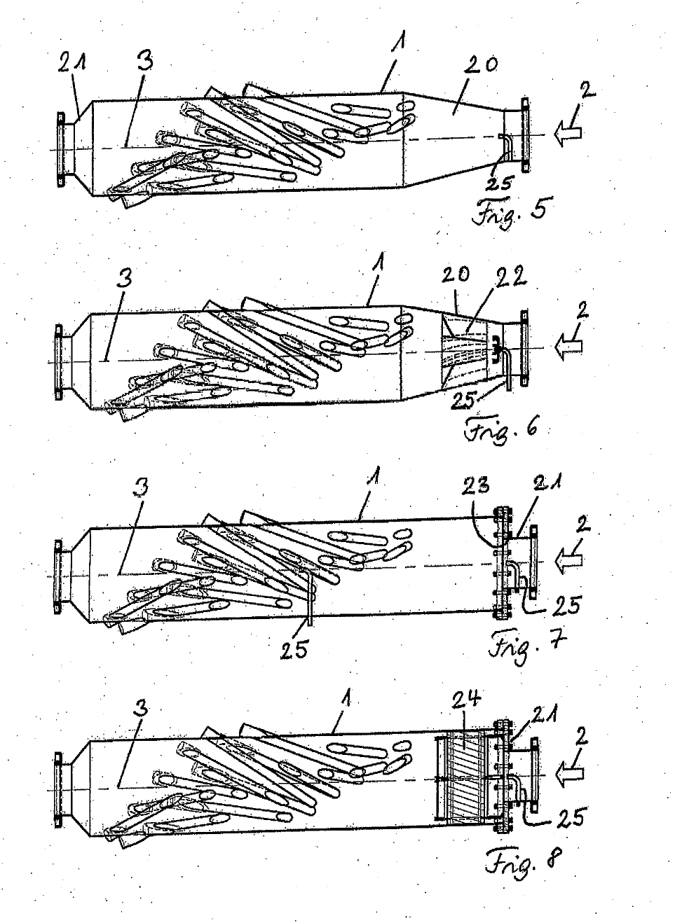

FIG. 5 shows a reactor having helically arranged UV emitters and a continuous diameter change in the area of the inlet 20 and the outlet 21 in a cross-section from the side. The arrangement of the emitters corresponds to those in FIGS. 3 and 4 and is described further above. The continuous diameter change causes a continuous widening of the flow at the inlet and hence a slowing of the flow which with sufficiently low speed remains almost laminar. Such an arrangement can be advantageous with pre-mixed media. FIG. 6 shows a reactor similar to FIG. 5 having a guide vane arrangement 22 in the inlet area 21, which provides turbulence and hence intermixing of the reagents present in the medium. The arrangement is particularly effective if the swirl direction of the guide vane arrangement 22 is oriented against the swirl direction of the helically arranged emitters.

FIG. 7 shows a reactor having a discontinuous cross-section change in the inlet area, which due to the whirl induced in the area of the discontinuity 23 causes intermixing.

Finally, FIG. 8 shows a reactor having a built-in device on the inlet side 21 for homogenising the flow. Such devices are known from chemical engineering as packings in columns. They cause components of the medium which were added upstream to be very extensively intermixed and provide an almost laminar, homogeneous flow which then encounters the UV emitters located downstream.

In operation, water can flow through the reactor in the direction of flow. At the inlet to the reactor, a liquid or gaseous oxidation means can be added via a dosing lance 25. The helix arrangement of the emitters causes the oxidation means, as it flows through the reactor, to be mixed homogeneously with the water flow and, at the same time, the oxidation reactions are triggered by the effect of the UV light. When using gaseous oxidation means, the reactor is advantageously arranged vertically and is flowed through from the bottom up. A distribution of gas with fine bubbles is hereby maintained for as long as possible. Since the UV radiation also affects the gas phase, reactions can also be brought about in the gas phase. For some processes, this can be of great significance, since gas phase reactions often take place at reaction speeds at higher orders of magnitude.

With oxidation reactions, controlling the pH value during the reaction is advantageous. This can be achieved via additional dosing lances, by means of which the corresponding reagents are added. The helix structure of the emitters also here causes the added chemicals to be homogeneously mixed into the flow.

Non-aqueous media can also be worked with. Thus, for example, a reaction can be triggered in an organic chemical by the effect of UV light. The use of a reactor is even advantageous with a single phase medium because uniform irradiation and hence a uniformly high conversion in the reaction are achieved by the homogeneous mixture. A further chemical can be added in the reactor inlet. In the reactor, the chemicals are then intermixed when flowing through by the helix structure, while at the same time a reaction is brought about by the UV light. The reactor can also be operated with gaseous media. An application, as an example, is the polymerisation from the gas phase, which is caused by UV light. Here, the helix structure provides a condensation surface, in order to deposit emerging fluid phases.

Certain oxidation processes require a photocatalyst in particle form. When such a particulate photocatalyst is used, the helix structure causes a homogeneous particle distribution to be maintained in the flow during UV irradiation.

By using the mixing device in the inlet corresponding to FIG. 6 or, in particular, to FIG. 8, a further homogenisation of the flow and a shortening of the overall length are achieved. In this way, particularly poorly intermixable chemicals can also already be pre-mixed. The helix structure of the UV reactor can then maintain the mixture, mixed on the inlet side, during the UV irradiation and counteract unmixing. This can advantageously be employed for the UV irradiation of an aqueous-organic two-phase flow.

1.-14. (canceled)

15. An ultraviolet (UV) reactor for carrying out chemical reactions in a pumpable medium by means of UV radiation, the reactor comprising a reactor chamber through which the medium can flow in a direction of flow from an inlet to an outlet, wherein the reactor chamber is penetrated by a plurality of UV transparent jacket tubes, which are arranged one behind the other in the direction of flow, and a plurality of UV emitters are arranged within the jacket tubes for emitting UV radiation into the reactor chamber, and wherein the jacket tubes are arranged one behind the other and are staggered against one another at an angle α in the circumferential direction of the reactor chamber.

16. The reactor according to claim 15, wherein the angle α is 15° to 45°.

17. The reactor according to claim 16, wherein the angle α is 30°.

18. The reactor according to claim 15, wherein the jacket tubes are inclined at an angle β of 30° to 70° with respect to the radial direction of the reactor chamber.

19. The reactor according to claim 15, wherein at least two groups of jacket tubes are provided, wherein each jacket tube of a first jacket tube group is arranged next to a jacket tube of the second jacket tube group with respect to the centre axis of the reactor chamber, and wherein the groups in each case form a helical row.

20. The reactor according to claim 15, wherein the jacket tubes are arranged at a distance from the centre axis of the reactor chamber.

21. The reactor according to claim 19, wherein the at least two groups of jacket tubes are at different distances from the centre axis, such that the first jacket tube group is a first distance away from the centre axis of the reactor chamber and the second jacket tube group is a second distance away from the centre axis of the reactor chamber.

22. The reactor according to claim 21, wherein the first distance is greater than the second distance.

23. The reactor according to claim 21, wherein the first jacket tube group is aligned at an angle β of 50° to 70° to the radial direction and the second jacket tube group is aligned at an angle β of 30° to 49° to the radial direction.

24. The reactor according to claim 21, wherein the first distance is more than 50% of the radius of the reactor chamber and the second distance is less than 50% of the radius of the reactor chamber.

25. The reactor according to claim 24, wherein the first distance is 75% of the radius of the reactor chamber and the second distance is 20% of the radius of the reactor chamber.

26. The reactor according to claim 24, wherein the axial distance within a jacket tube group is varied, the first jacket tube group having an averaged axial first distance which varies by +/−10% of the radius, while the second jacket tube group has an averaged axial second distance, which likewise varies by +/−10% of the radius of the reactor chamber.

27. The reactor according to claim 19, wherein each of the groups of jacket tubes comprises a total 12 jacket tubes.

28. The reactor according to claim 15, wherein a means for intermixing the medium is arranged at the inlet to the reactor chamber.

29. The reactor according to claim 15, wherein a means for feeding oxygen, ozone and/or H₂O₂ or other oxidation means into the medium is arranged at the inlet to the reactor chamber.

30. Use of the reactor described in claim 15 for the oxidative degradation of inert hydrocarbons.
